(19) United States
(12) Patent Application Publication
(10) Pub. No.: US 2025/0134562 A1
(43) Pub. Date: May 1, 2025

(54) SUTURE HOLE WITH FUNNEL ON BONE PLATE

(71) Applicant: DEPUY SYNTHES PRODUCTS, INC., Raynham, MA (US)

(72) Inventors: Frederic Gueissaz, Reconvilier (CH); Henri Defossez, Neuchatel (CH); Martin Altmann, Zuchwil (CH); Roger Koch, Aarau (CH)

(21) Appl. No.: 18/497,618

(22) Filed: Oct. 30, 2023

(51) Int. Cl. A61B 17/80; A61B 17/04

(52) U.S. Cl. CPC A61B 17/80; A61B 17/0401; A61B 2017/0414

(57) ABSTRACT

A bone T-plate, including: a plate head with a plurality of head screw holes, a plurality of suture holes, and a plurality of funnel openings, wherein the plurality of head screw holes are spaced along a length of the plate head, the plurality of funnel openings are each associated with one of the plurality of suture holes, the plurality of funnel openings are on a lower surface of the plate head that is configured to contact a bone, the plurality of funnel openings have a wide portion at a first edge of the plate head, a narrow portion at its associated suture hole, and extend from the first edge to its associated suture hole, and the plurality of suture holes are spaced along the length of the plate head and each arranged in position aligned between two of the plurality of head screw holes along the length of the plate head; and a plate shaft extending from a second edge of the plate head, wherein the second edge is opposite the first edge.

### FIELD OF THE DISCLOSURE

Various exemplary embodiments disclosed herein relate to bone plates having suture holes with funnels.

### BACKGROUND

Various types of bone plates are used during reduction and internal fixation of bone fragments. In addition to securing a bone plate to a patient's bone, sutures may be used to attach soft tissue to the bone plate. Such bone plates may take various sizes and shapes including a T-shape.

### SUMMARY

A summary of various exemplary embodiments is presented below.

Various embodiments relate to a bone T-plate, including: a plate head with a plurality of head screw holes, a plurality of suture holes, and a plurality of funnel openings, wherein the plurality of head screw holes are spaced along a length of the plate head, the plurality of funnel openings are each associated with one of the plurality of suture holes, the plurality of funnel openings are on a lower surface of the plate head that is configured to contact a bone, the plurality of funnel openings have a wide portion at a first edge of the plate head, a narrow portion at its associated suture hole, and extend from the first edge to its associated suture hole, and the plurality of suture holes are spaced along the length of the plate head and each arranged in position aligned between two of the plurality of head screw holes along the length of the plate head; and a plate shaft extending from a second edge of the plate head, wherein the second edge is opposite the first edge.

Various embodiments are described, wherein the plurality of suture holes have an edgeless suture hole lower border and an edgeless suture hole upper border.

Various embodiments are described, wherein the edgeless suture hole lower border and edgeless suture hole upper border rounded or chamfered.

Various embodiments are described, wherein the plate head is bendable at the plurality of funnel openings.

Various embodiments are described, wherein the plurality of head screw holes are threaded and configured to accept one of locking screws or variable angle screws.

Various embodiments are described, wherein the T-plate has a thickness of approximately 2.0 mm.

Various embodiments are described, wherein the T-plate has a thickness that is less than or equal to approximately 2.0 mm.

Various embodiments are described, wherein the T-plate has a thickness of approximately 1.7 mm.

Various embodiments are described, wherein the T-plate has a thickness that is less than or equal to approximately 1.7 mm.

Various embodiments are described, wherein the T-plate has a thickness of approximately 1.5 mm.

Various embodiments are described, wherein the T-plate has a thickness that is less than or equal to approximately 1.5 mm.

Further various embodiments relate to a method for applying a bone T-plate wherein the bone T-plate includes: a plate head with a plurality of head screw holes, a plurality of suture holes, and a plurality of funnel openings, wherein the plurality of head screw holes are spaced along a length of the plate head, the plurality of funnel openings are each associated with one of the plurality of suture holes, the plurality of funnel openings are on a lower surface of the plate head that is configured to contact a bone, the plurality of funnel openings have a wide portion at a first edge of the plate head, a narrow portion at its associated suture hole, and extend from the first edge to its associated suture hole, and the plurality of suture holes are spaced along the length of the plate head and each arranged in position aligned between two of the plurality of head screw holes along the length of the plate head; and a plate shaft extending from a second edge of the plate head, wherein the second edge is opposite the first edge; the method including: bending the plate head along one of the plurality of funnel openings to conform with a bone; securing soft tissue to the T-plate by applying a suture through soft tissue and through one of the plurality of suture holes and its associated funnel; and securing the T-plate to the bone by placing a plurality of screws though some of the plurality of head screw holes.

Various embodiments are described, further comprising inserting a K-wire through one of the plurality of suture holes into the bone to temporarily secure the T-plate to the bone.

The foregoing has outlined rather broadly the features and technical advantages of examples according to the disclosure in order that the detailed description that follows may be better understood. Additional features and advantages will be described hereinafter. The conception and specific examples disclosed may be readily utilized as a basis for modifying or designing other structures for carrying out the same purposes of the present disclosure. Such equivalent constructions do not depart from the scope of the appended claims. Characteristics of the concepts disclosed herein, both their organization and method of operation, together with associated advantages will be better understood from the following description when considered in connection with the accompanying figures. Each of the figures is provided for the purposes of illustration and description, and not as a definition of the limits of the claims.

### BRIEF DESCRIPTION OF DRAWINGS

So that the above-recited features of the present disclosure can be understood in detail, a more particular description, briefly summarized above, may be had by reference to aspects, some of which are illustrated in the appended drawings. It is to be noted, however, that the appended drawings illustrate only certain typical aspects of this disclosure and are therefore not to be considered limiting of its scope, for the description may admit to other equally effective aspects. The same reference numbers in different drawings may identify the same or similar elements.

FIG. 1A illustrates a top perspective view of a T-plate.

FIG. 1B illustrates a bottom perspective view of the T-plate.

FIG. 1C illustrates a bottom view of the T-plate.

FIG. 1D illustrates a top view of the T-plate.

FIG. 1E illustrates a top perspective view of a head of the T-plate.

FIG. 1F illustrates a bottom perspective view of the head of the T-plate.

FIG. 1G illustrates a broad end view of the T-plate.

FIG. 1H illustrates bottom perspective view of a suture hole with funnel of the T-plate.

FIGS. 2A and 2B illustrate top perspective and bottom perspective views of another embodiment of a T-plate.

FIG. 3 illustrates the placement of a curved needle with an attached suture through a suture hole.

FIG. 4 illustrates the placement of a curved needle with an attached suture through soft tissue and a suture hole.

FIG. 5 illustrates the suture fed through the suture hole.

### DETAILED DESCRIPTION

Various aspects of the disclosure are described more fully hereinafter with reference to the accompanying drawings. This disclosure may, however, be embodied in many different forms and should not be construed as limited to any specific structure or function presented throughout this disclosure. Rather, these aspects are provided so that this disclosure will be thorough and complete, and will fully convey the scope of the disclosure to those skilled in the art. Based on the teachings herein one skilled in the art should appreciate that the scope of the disclosure is intended to cover any aspect of the disclosure disclosed herein, whether implemented independently of or combined with any other aspect of the disclosure. For example, an apparatus may be implemented or a method may be practiced using any number of the aspects set forth herein. In addition, the scope of the disclosure is intended to cover such an apparatus or method which is practiced using other structure, functionality, or structure and functionality in addition to or other than the various aspects of the disclosure set forth herein. It should be understood that any aspect of the disclosure disclosed herein may be embodied by one or more elements of a claim.

Several aspects of bone plates with suture holes having a funnel will now be presented with reference to various apparatuses and techniques. These apparatuses and techniques will be described in the following detailed description and illustrated in the accompanying drawings by various blocks, components, steps, methods, and/or the like (collectively referred to as “elements”).

This disclosure relates generally to bone plates including suture holes having a funnel where the suture hole accepts sutures that are attached to soft tissue.

When bones are damaged through trauma, disease, distraction osteogenesis, or orthognathic surgery, the defect is typically reduced, and bone fixation plates are commonly applied to the bone to ensure union in the desired position. Bone plates are typically made from a rigid material, such as titanium or stainless steel, and include fixation holes that are sized to be driven through the fixation holes and into the underlying bone to secure the bone plate to the bone. One common bone screw used in such application can be referred to as a compression screw. Compression screws can have unthreaded heads and threaded shafts. Accordingly, the compression screw can be driven through the plate fixation hole and into the underlying bone until the head applies a compression force against the bone plate toward the underlying bone. Another common bone screw used in such applications is generally referred to as a locking screw. Locking screws have threaded heads and threaded shafts. Accordingly, the locking screw can be driven through the plate fixation hole and into the underlying bone until the head thread mates with the thread in the fixation hole of the plate. Thus, the head of the locking screw does not apply a compressive force against the bone plate toward the underlying bone.

Conventionally, locking screws were inserted through the screw hole along the central screw hole axis in order to ensure that the threaded screw head mates with the plate in the threaded fixation hole. Recently, however, bone plates have been developed having threaded fixation holes that are configured to receive locking screws at different trajectories within a range of trajectories whereby the bone plate threadedly mates with the locking screw head in the threaded hole.

Further, when a bone plate is used to treat and stabilize a fracture of a bone, it may be necessary to attach and anchor adjacent soft tissue to the bone. In some cases this may done by placing a suture through the soft tissue and passing the suture through a screw hole on the bone plate to secure the soft tissue. This means that the screw hole used is not available to accept a screw to secure the bone plate to the bone. Further, the threads that may be present in the screw hole are sharp and may sever the suture. Separate suture holes may be used instead to accept sutures used to secure soft tissue. Such suture holes may not sever the suture and allow for all of the screw holes in the bone plate to be used, but as the plate is secured to the bone it will trap the suture between the bone and the bone plate which may precludes the tightening of the suture and poses an increased risk of suture breaking. In order to overcome these challenges, a suture hole combined with a funnel opening on the bottom side of the plate that contacts the bone will now be described.

Generic T-plates are often used for the fixation of sutures in the metaphyseal area. Such T-plates may further be thin enough to allow for them to be bent to the contour of the bone where it will be placed. T-plates may be used in many other applications as well.

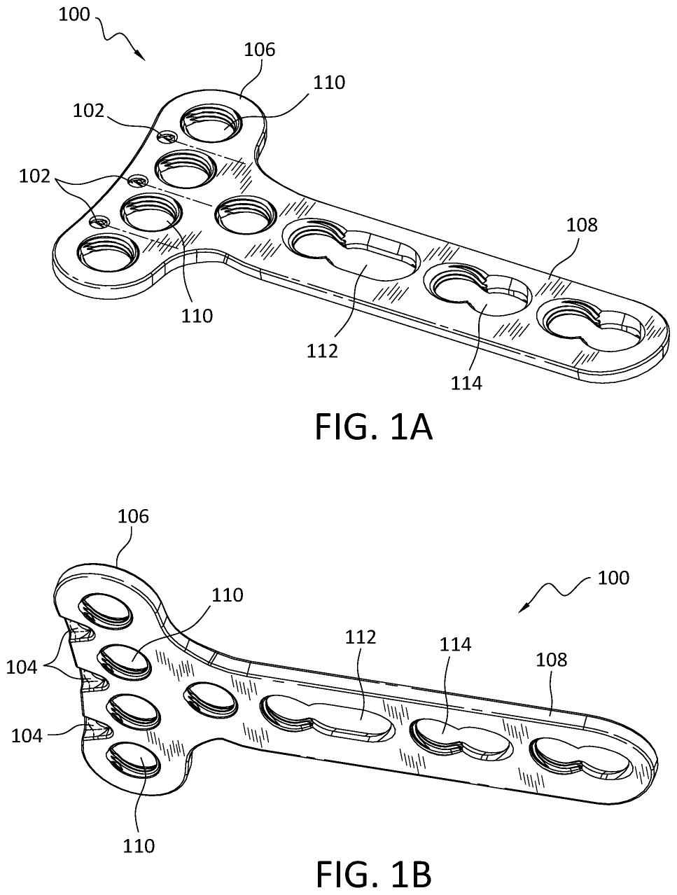

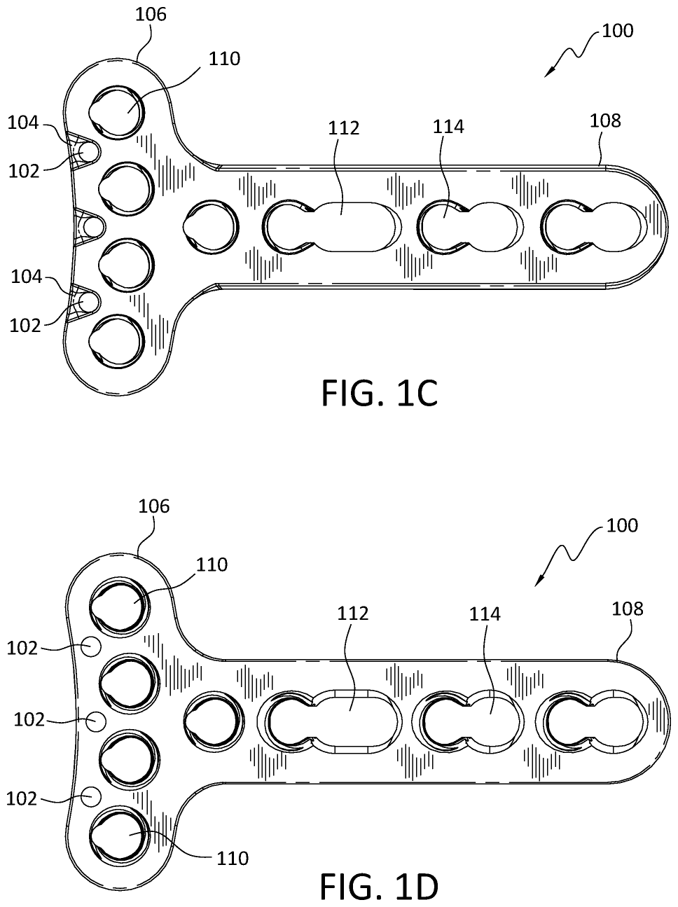

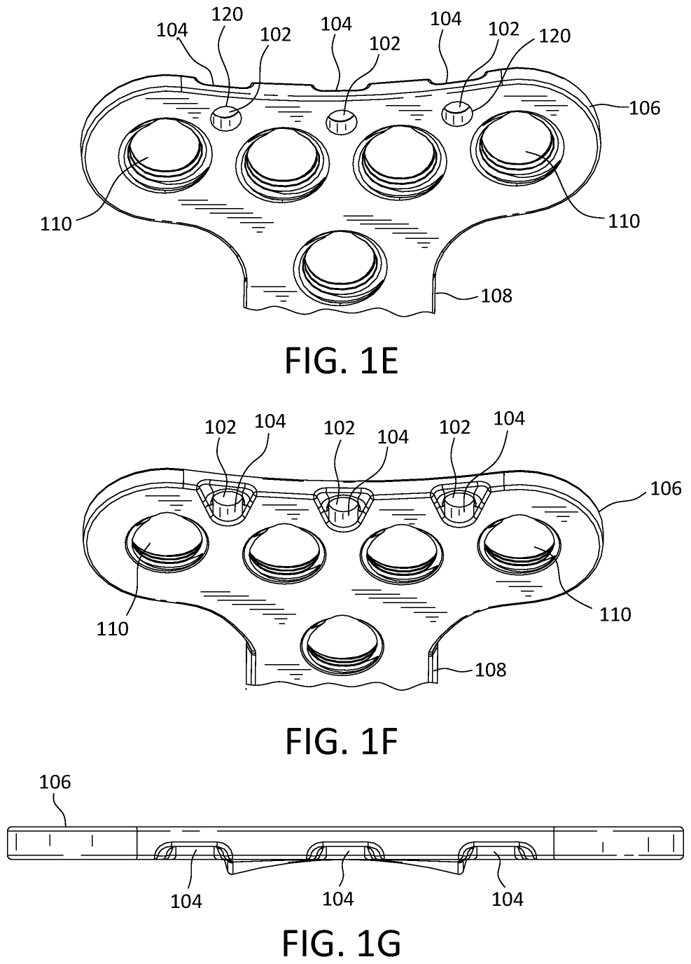

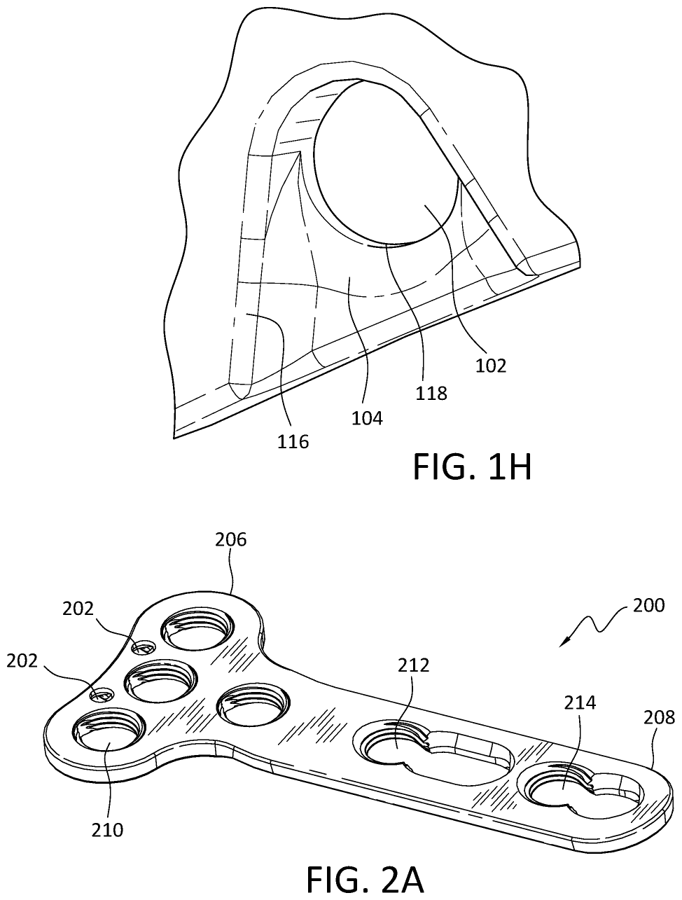

FIG. 1A illustrates a top perspective view of a T-plate. FIG. 1B illustrates a bottom perspective view of the T-plate. FIG. 1C illustrates a bottom view of the T-plate. FIG. 1D illustrates a top view of the T-plate. FIG. 1E illustrates a top perspective view of a head of the T-plate. FIG. 1F illustrates a bottom perspective view of the head of the T-plate. FIG. 1G illustrates a broad end view of the T-plate. FIG. 1H illustrates bottom perspective view of a suture hole and funnel of the T-plate. The T-plate 100 includes a plate head 106 and a plate shaft 108. In this example, the plate head 106 includes four head screw holes 110 and three suture holes 102. The plate head 106 may include other numbers of head screw holes 110 and suture holes 102. The head screw holes 110 may be threaded to accommodate locking screws or variable angle screws. In other embodiments the head screw hole 110 may have no threads.

Both the head screw holes 110 and suture holes 102 may be spaced along a length of the plate head. Further, the plurality of suture holes 102 are arranged closer to the edge of the plate head 106 and the head screw holes 110. There will typically be fewer suture holes 102 than the head screw holes 110, so that the suture holes 102 can be placed near the edge of the plate head 106 but aligned between the head screw holes 110, i.e., the suture holes 102 are aligned with the portion of the plate head 106 that is between the head screw holes 110 (as indicated by the dashed lines in FIG. 1A).

The plate shaft 108 may include elongated combi-hole 112 and combi-hole 114 that may receive compression, locking, or variable angle bone screws to secure the T-plate 100 to a bone. While the length of the plate shaft 108 is shown as longer than the length of the plate head 106 (the length of the plate head 106 and plate shaft 108 can be substantially perpendicular to one another), any length for plate head 106 and plate shaft 108 may be used depending on the specific application of the T-plate 100. Further, the plate shaft 108 is shown as extending generally from the center of the plate head 106, but the plate shaft 108 may also extend from other parts of the plate head 106. Also, the plate shaft 108 is shown as extending generally perpendicular to the plate head 106, but the plate shaft 108 may also extend at other angles depending on the application of the T-plate 100.

The plate head 106 includes a funnel opening 104 associated with each of the suture holes 102. The funnel opening 104 is on the bottom side of the plate head 106 that contacts the bone. The funnel opening 104 has a generally triangular or funnel shape that starts at the suture hole 102 and widens as it extends towards an edge of the plate head 106, so the funnel opening 104 has a narrow portion at the suture holes 102 and a wide portion at the edge of the plate head 106. The funnel opening 104 includes a funnel side surface 116. The depth of the funnel may be about 50% of thickness of the T-plate 100, but other depths may be used as well. The depth may be greater than the thickness of a suture to be used to suture soft tissue to the T-plate 100 so that the suture is not trapped by the T-plate 100 against the bone.

The suture hole 102 may have a suture hole lower border 118 that is edgeless, for example, rounded or chamfered. The edgeless shape of the suture hole lower border 118 prevents suture hole lower border 118 of the suture hole 102 from severing a suture fed through the suture hole 102. Likewise, the suture hole 102 may have a suture hole upper border 120 that may also be edgeless, for example, rounded or chamfered to prevent the suture hole upper border 120 from severing a suture fed through the suture hole 102.

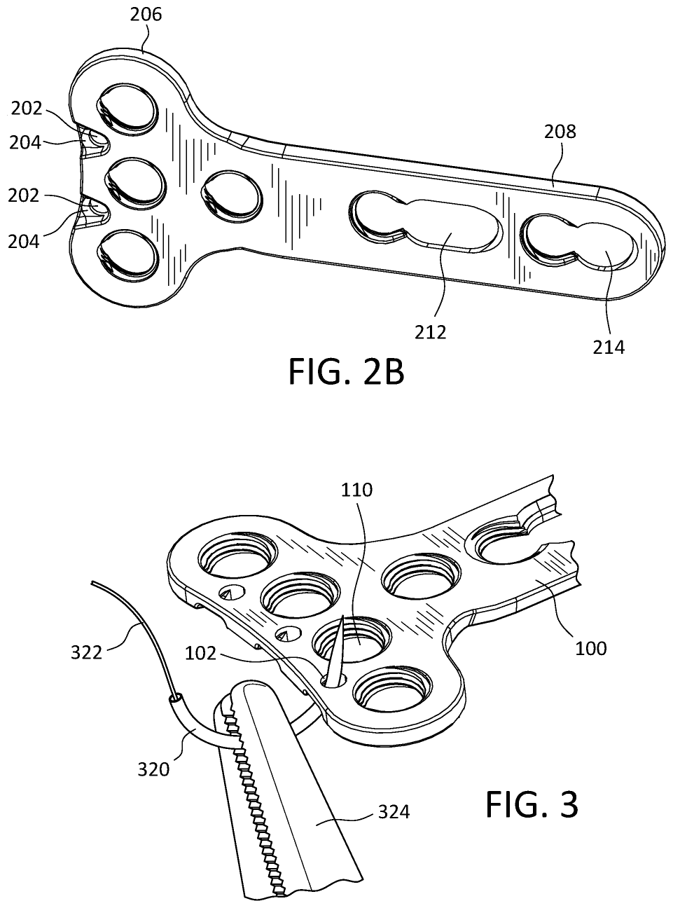

FIGS. 2A and 2B illustrate top perspective and bottom perspective views of another embodiment of a T-plate. The T-plate 200 includes a plate head 206 and plate shaft 208. The plate head 206 includes suture holes 202 and head screw holes 210. In this example, there are three head screw holes 210 and two suture holes 202. The plate head 206 includes a funnel opening 204 associated with each of the suture holes 202. The plate shaft 208 may include elongated screws hole 212 and combi-hole 214. The T-plate 200 is of a similar design as the T-plate 100 of FIG. 1, but with different numbers of the various holes.

FIG. 3 illustrates the placement of a suture through a suture hole. A needle 320 is attached to a suture 322. Pliers 324 may be used to grip the needle 320 and to insert the needle 320 through the suture hole 102 of the T-plate 100. The needle 320 can then be fed through the suture hole 102 to pull the suture through the suture hole 102. Not shown in FIG. 3 is soft tissue through which the needle 320 and suture 322 would be placed to secure the soft tissue to the T-plate 100. It noted that the suture holes 102 may also be used as K-wire holes to help position and preliminarily position the T-plate to the bone.

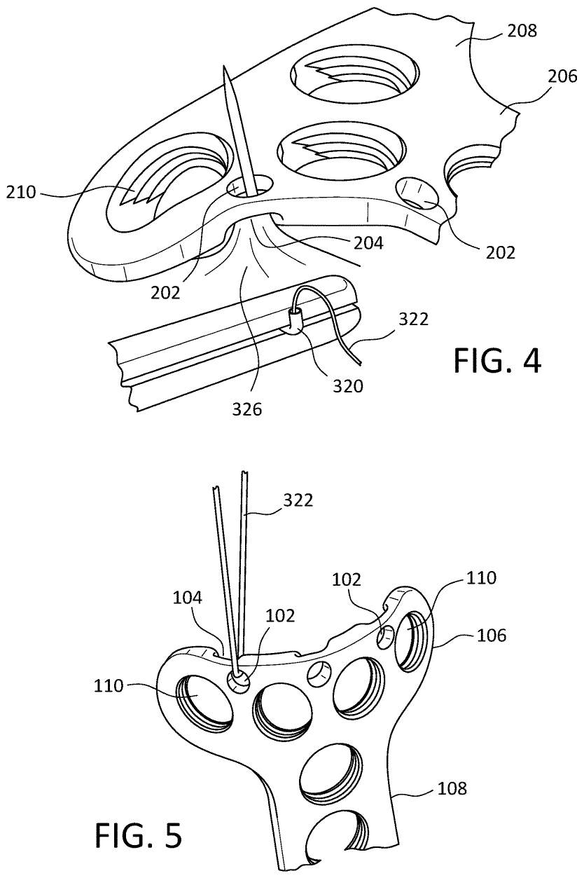

FIG. 4 illustrates the placement of a suture through soft tissue and a suture hole. The tip of the needle 320 is inserted from the bottom side of the plate through the suture hole 202. The suture 322 can then be pulled through the suture hole 202 and tied off to secure the soft tissue 326 to the T-plate 200. Because of the funnel opening 204, the suture is able to move freely through the suture hole 202 and funnel opening 204 without being trapped by the T-plate 200 against the bone when the T-plate 200 is secured to the bone.

FIG. 5 illustrates the suture fed through the suture hole 102. In can be seen that the suture 322 runs through the funnel opening 104 to prevent the suture 322 from being trapped between the T-plate 100 and the bone.

The T-plate 100 and T-plate 200 can both be bent to match the bone anatomy where the T-plate is being placed. This allows for a generic T-plate that can then be adapted to the patient's specific anatomy. The bendability of the plate head are facilitated by the thinness of the plates and the material selection. Because the head of the T-plate is thinner at the funnel opening, that portion of the head of the T-plate will be easier to bend. Because the funnel openings are arranged between the head screw holes, it will be easier to implement a bend between the head screw holes and to thereby prevent bending of the head screw holes. Hence the location of the funnel opening not only provides the benefit of allowing the suture to move freely, but also to facilitate bending the plate head in a specific desired location. The plate may be bent using any technique for bending the plate according to the applicable surgical technique guide.

The T-plate is designed with the lowest possible thickness considering the functionality according to the indication. A low thickness of the plate has the advantage of a low protrusion of the plate and the screws. Accordingly, the T-plate may have a thickness of about 2.0 mm, 1.9 mm, 1.8 mm, 1.7 mm, 1.6 mm, or 1.5 mm. Further T-plate may have a thickness in a range that is less than or equal to about 2.0 mm, 1.9 mm, 1.8 mm, 1.7 mm, 1.6 mm, or 1.5 mm.

The T-plate may be applied to a bone using the following steps. After the location of the bone where the T-plate is to be placed is exposed, the surgeon will contour the T-plate to conform to the anatomy of the patient. This bending is done along one or more of the funnel openings. Next, the T-plate is placed on the bone. The T-plate in some situations may be temporarily attached to the bone, for example, by using a K-wire through one of the suture holes or dedicated K-wire holes. At this point the surgeon may use a needle with an attached suture to secure the soft tissue to the T-plate by threading the suture through the soft tissue and through one of the suture holes and its associated funnel, and then tying the suture. Then the T-plate may be secured to the bone by placing a plurality of screws through some of the head screw holes and the screw holes in the shaft.

The foregoing disclosure provides illustration and description but is not intended to be exhaustive or to limit the aspects to the precise form disclosed. Modifications and variations may be made in light of the above disclosure or may be acquired from practice of the aspects.

Unless stated otherwise, terms such as “first” and “second” are used to arbitrarily distinguish between the elements such terms describe. Thus, these terms are not necessarily intended to indicate temporal or other prioritization of such elements.

It should be appreciated by those skilled in the art that any block diagrams herein represent conceptual views of illustrative hardware embodying the principles of the aspects.

While each of the embodiments are described above in terms of their structural arrangements, it should be appreciated that the aspects also cover the associated methods of using the embodiments described above.

Unless otherwise indicated, all numbers expressing parameter values and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by embodiments of the present disclosure. As used herein, “about” may be understood by persons of ordinary skill in the art and can vary to some extent depending upon the context in which it is used. If there are uses of the term which are not clear to persons of ordinary skill in the art, given the context in which it is used, “about” may mean up to plus or minus 10% of the particular term or may be defined by normal manufacturing tolerances and processes.

Even though particular combinations of features are recited in the claims and/or disclosed in the specification, these combinations are not intended to limit the disclosure of various aspects. In fact, many of these features may be combined in ways not specifically recited in the claims and/or disclosed in the specification. Although each dependent claim listed below may directly depend on only one claim, the disclosure of various aspects includes each dependent claim in combination with every other claim in the claim set. A phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

No element, act, or instruction used herein should be construed as critical or essential unless explicitly described as such. Also, as used herein, the articles “a” and “an” are intended to include one or more items and may be used interchangeably with “one or more.” Furthermore, as used herein, the terms “set” and “group” are intended to include one or more items (e.g., related items, unrelated items, a combination of related and unrelated items, and/or the like), and may be used interchangeably with “one or more.” Where only one item is intended, the phrase “only one” or similar language is used. Also, as used herein, the terms “has,” “have,” “having,” and/or the like are intended to be open-ended terms. Further, the phrase “based on” is intended to mean “based, at least in part, on” unless explicitly stated otherwise.

1. A bone T-plate, comprising:
a plate head with a plurality of head screw holes, a plurality of suture holes, and a plurality of funnel openings, wherein
the plurality of head screw holes are spaced along a length of the plate head,
the plurality of funnel openings are each associated with one of the plurality of suture holes,
the plurality of funnel openings are on a lower surface of the plate head that is configured to contact a bone,
the plurality of funnel openings have a wide portion at a first edge of the plate head, a narrow portion at its associated suture hole, and extend from the first edge to its associated suture hole, and
the plurality of suture holes are spaced along the length of the plate head and each arranged in position aligned between two of the plurality of head screw holes along the length of the plate head; and
a plate shaft extending from a second edge of the plate head, wherein the second edge is opposite the first edge.

2. The bone T-plate of claim 1, wherein the plurality of suture holes have an edgeless suture hole lower border and an edgeless suture hole upper border.

3. The bone T-plate of claim 2 where the edgeless suture hole lower border and edgeless suture hole upper border rounded or chamfered.

4. The bone T-plate of claim 1, wherein the plate head is bendable at the plurality of funnel openings.

5. The bone T-plate of claim 1, wherein the plurality of head screw holes are threaded and configured to accept one of locking screws or variable angle screws.

6. The bone T-plate of claim 1, wherein the bone T-plate has a thickness of approximately 2.0 mm.

7. The bone T-plate of claim 1, wherein the bone T-plate has a thickness that is less than or equal to approximately 2.0 mm.

8. The bone T-plate of claim 1, wherein the bone T-plate has a thickness of approximately 1.7 mm.

9. The bone T-plate of claim 1, wherein the bone T-plate has a thickness that is less than or equal to approximately 1.7 mm.

10. The bone T-plate of claim 1, wherein the bone T-plate has a thickness of approximately 1.5 mm.

11. The bone T-plate of claim 1, wherein the bone T-plate has a thickness that is less than or equal to approximately 1.5 mm.

12. A method for applying a bone T-plate wherein the bone T-plate comprises:
a plate head with a plurality of head screw holes, a plurality of suture holes, and a plurality of funnel openings, wherein
the plurality of head screw holes are spaced along a length of the plate head,
the plurality of funnel openings are each associated with one of the plurality of suture holes,
the plurality of funnel openings are on a lower surface of the plate head that is configured to contact a bone,
the plurality of funnel openings have a wide portion at a first edge of the plate head, a narrow portion at its associated suture hole, and extend from the first edge to its associated suture hole, and
the plurality of suture holes are spaced along the length of the plate head and each arranged in position aligned between two of the plurality of head screw holes along the length of the plate head; and
a plate shaft extending from a second edge of the plate head, wherein the second edge is opposite the first edge;
the method comprising:
bending the plate head along one of the plurality of funnel openings to conform with a bone;
securing soft tissue to the bone T-plate by applying a suture through soft tissue and through one of the plurality of suture holes and its associated funnel; and
securing the bone T-plate to the bone by placing a plurality of screws though some of the plurality of head screw holes.

13. The method of claim 12, further comprising inserting a K-wire through one of the plurality of suture holes into the bone to temporarily secure the bone T-plate to the bone.
